(19) United States
(12) Patent Application Publication
(10) Pub. No.: US 2008/0199946 A1
(43) Pub. Date: Aug. 21, 2008

(54) Biochip

(76) Inventors: Chung-Cheng Chang, Keelung (TW); Jau-Der Chen, Keelung (TW); Pei-Tai Chen, Keelung (TW)

Correspondence Address: LIN & ASSOCIATES INTELLECTUAL PROPERTY, INC., P.O. BOX 2339, SARATOGA, CA, 95070-0339, US

(21) Appl. No.: 11/675,637

(22) Filed: Feb. 16, 2007

(51) Int. Cl. C12M 1/34

(52) U.S. Cl. 435/287.2

(57) ABSTRACT

The present invention relates to a biochip for nucleic acid hybridization. The biochip of the present invention comprises a hybridization chamber which is in the form of a cavity, a porous matrix pressed in the hybridization chamber; and at least one first circulation hole and at least one second circulation hole which are communicated with the hybridization chamber so that the reaction solution flows in the at least one first circulation hole and flows out the at least one second circulation hole through the pores of the porous matrix. The hybridization reaction area is increased by flowing the reaction solution through the pores of the membrane, which enable the reaction sensitivity to be increased. The diffusion distance for the reaction molecules is decreased due to the limited inside space of the membrane, and thereby the hybridization time is shortened.

### BACKGROUND OF THE INVENTION

1. Field of the Invention

The present invention relates to a biochip, and particularly relates to a biochip for nucleic acid hybridization.

2. The Prior Arts

The hybridization method in which the nucleic acid probe is hybridized to the target nucleic acid is one of the most common analytical techniques to confirm whether the target DNA has the desired gene or nucleic acid sequence or not. Conventionally, the blotting process used in hybridization analysis is to transfer the target nucleic acid to a substrate such as membrane, and then the nucleic acid probe with specificity is applied for hybridization, and then the color, chemiluminescence, or radioactivity exhibited by the labeled molecules in the nucleic acid probe is detected whereby it is possible to judge whether a target base sequence is present in the target nucleic acid or not.

One of the hybridization techniques used today is the “Southern blotting”, in which the target DNA is transferred from an electrophores gel to a membrane, and then hybridized with a probe. When used with RNA target the method is called “Northern blotting”. In the other methods, in accordance with the dropping area the target nucleic acid is directly dropped onto the membrane by dot blotting, slot blotting, or spot blotting. In the dot blotting, slot blotting, and spot blotting method, the nucleic acid can be directly blotted onto a substrate without the transfer process of electrophoresis. Therefore, the analysis time for target nucleic acid is reduced. The Blotting method can be used in the qualitative analysis by batch.

In the conventional blotting assay, after the target nucleic acid is dropped onto the surface of the membrane, the nucleic acid is permanently attached to the membrane by cross-linking using heating or UV radiation so that the target nucleic acid will remain on the film when being washed after the step of hybridization with a probe. Because the target nucleic acid is dropped onto a wet surface of a membrane, and then diffuses and adsorbs on the wet surface under the known working conditions. Therefore, most of the target nucleic acid is only firmly absorbed on the membrane surface and the nearby pores thereof, so that the numbers of nucleic acid molecules firmly absorbed are limited, and thus the produced hybridization signal is relatively weak. As a result, if the amount of the nucleic acid sample is not enough or the nucleic acid sample has a long molecular chain, the reaction sensitivity will be greatly reduced. When the probe hybridization solution containing the blocking reagent is added to the membrane, the nucleic acid probe can only diffuse on the surface of the membrane as the target nucleic acid do, and find out the target nucleic acid for hybridization in Brownian movement. Therefore, the nucleic acid hybridization reaction takes more processes to be accomplished, and the reaction time is more than 10 hours. Therefore, the hybridization results cannot be obtained in a short time. In addition, it is not economical for the qualitative analysis of the nucleic acids if the hybridization assay takes long time and needs lots of reagents. Therefore, there is a need for the development of a simple blotting device and a blotting method to reduce the process steps and time for hybridization assay and to reduce the background noise, and thus such a blotting device and such a blotting method can be applied to the detection for simple or batch process.

### SUMMARY OF THE INVENTION

In order to solve the time-consuming problem in hybridization assay when the nucleic acid probe is base-paired with the target nucleic acid by Brownian motion, and in order to increase the amount of target nucleic acid firmly absorbed on the surface and the inside of the pores of the membrane for increasing base pairing probabilities and reaction sensitivity, the present invention provides a biochip for nucleic acid hybridization assays. In the present invention, the inside space of the biochip is limited so that the target nucleic acid and the nucleic acid probe can rapidly diffuse into the micropores of the substrate in a very short time and are base paired with each other when the hybridization solution enters the inside of the substrate. Furthermore, under the condition of pressurizing the fluid, the target nucleic acid and the nucleic acid probe can move rapidly, and because the adsorption and reaction area of the target nucleic acid and the nucleic acid probe are enlarged when they flow into the inside of the membrane, the number of base pairing is increased, which can enhance the detection sensitivity. Meanwhile, the base pairing between the target nucleic acid and the nucleic acid probe is speeded up due to the flowing movement of the nucleic acid molecules. Moreover, because the washing solution can be deeply flushed into the inside of the membrane and washes away the probe molecules unspecifically bound to the membrane, and thereby the cleanness is improved and the reaction background level is reduced.

In order to achieve the above objectives , the present invention provides a biochip, comprising: a hybridization chamber which is in the form of a cavity, a porous matrix pressed in the hybridization chamber; and at least one first circulation hole and at least one second circulation hole which are communicated with the hybridization chamber so that the reaction solution flows in the at least one first circulation hole and flows out the at least one second circulation hole through the pores of the porous matrix. The biochip includes an upper substrate and a lower substrate, wherein the upper substrate and the lower substrate are stacked together one on top of the other to form the hybridization chamber therein, and the porous matrix is provided in the hybridization chamber, and at least one first circulation hole located at the upper substrate and at least one second circulation hole, which are communicated with the hybridization chamber, are provided on the top and the side of the hybridization chamber, respectively.

There is no specific limitation on the shape and the thickness of the hybridization chamber, and its shape and thickness can be changed with the porous matrix structure. An interstice with predetermined width is left between the porous matrix and the sidewall of the hybridization chamber so that the reaction solution can enter the porous matrix from the side thereof. In addition, the central top of the hybridization chamber is provided with a first circulation hole. There is no specific limitation on the position and the number of the first circulation hole. The first circulation hole, the second circulation hole, and the hybridization chamber can be further communicated with a microchannel, and the reaction solution can rapidly pass through the porous matrix by pressurizing the reaction solution via the microchannel, and thereby the reaction rate is increased. If the porous matrix is in a dry state before the reaction, the reaction solution can rapidly enter the inside of the porous matrix due to the capillary attraction of the pores of the membrane. The membrane has a pore diameter of 0.1 μm to 50 μm. The membrane can be a nylon membrane, a nitrocellulose membrane, or any other suitable membrane.

In the biochip of the present invention, the target nucleic acid can enter the hybridization chamber via one or more circulation holes and can be absorbed by the membrane therein. Because the target nucleic acid can enter the inside of the membrane, the number of the target nucleic acid molecules firmly adsorbed by the membrane is increased, which can enhance the detection sensitivity. After the target nucleic acid molecules are firmly adsorbed by the membrane, the nucleic acid probe solution enter the membrane pressed in the hybridization chamber via one or more circulation holes and anneal with the target nucleic acid. Because the nucleic acid probe can easily move in the pores of the membrane, it can anneal with the target nucleic acid in a very short time. Furthermore, the washing solution is flushed into the membrane via one or more circulation holes for washing. Because the nucleic acid probe can easily move in the pores of the membrane, the nucleic acid probe molecules unspecifically bound to the membrane can be easily and rapidly flushed out of the membrane. Therefore, the washing process is rapid and complete, the reaction time is shortened, and the background noise level is reduced.

The present invention provides a biochip, comprising an upper substrate and a lower substrate, which are stacked together one on top of the other. A hybridization chamber is provided between the upper substrate and the lower substrate, and a porous matrix is provided in the hybridization chamber. A plurality of little pillars protrude from an interface between a bottom of the upper substrate and the hybridization chamber wherein the ends of the little pillars are in contact with the surface of the porous matrix pressed in the hybridization chamber. In addition, at least one first circulation hole located at the upper substrate and at least one second circulation hole are provided on a top and a side of the hybridization chamber, respectively, wherein the second circulation hole is communicated with the interspace among the little pillars, so that the reaction solution is able to fill up the interspace among the little pillars via the second circulation hole and then enters the membrane. By using such little pillars, the area occupied by the reaction solution in the membrane is enlarged, and thereby the rate of the reaction solution that enters the membrane and its efficiency are increased. As a result, the reaction is rapid and complete.

The present invention provides a biochip comprising an upper substrate and a lower substrate, and the lower substrate can be a single-layer lower substrate or can be composed of a top substrate and a bottom substrate. The third circulation hole which is communicated with hybridization chamber can be provided in a single-layer lower substrate or in a lower substrate composed of a top substrate and a bottom substrate. The third circulation hole is further communicated with the second microchannel, and the second microchannel is further communicated with the fourth circulation hole so that the reaction solution can enter the membrane from the bottom of the lower substrate. Therefore, the reaction solution can enter the membrane from different flow paths.

Other objects, advantages, and novel features of the invention will become more apparent from the following detailed description when taken in conjunction with the accompanying drawings.

### BRIEF DESCRIPTION OF THE DRAWINGS

FIG. 1A is an exploded view of the biochip according to the first embodiment of the present invention;

FIG. 1B is a cross-section view after the biochip is assembled according to the first embodiment of the present invention;

FIG. 1C is a top view after the biochip is assembled according to the first embodiment of the present invention;

FIG. 1D is a schematic view of the solution flowing direction during the hybridization reaction according to the first embodiment of the present invention;

FIG. 1E is a top view of the biochip derived from the first embodiment of the present invention;

FIG. 2A is an exploded view of the biochip according to the second embodiment of the present invention;

FIG. 2B is a cross-section view after the biochip is assembled according to the second embodiment of the present invention;

FIG. 2C is a top view after the biochip is assembled according to the second embodiment of the present invention;

FIG. 2D is a schematic view of the solution flowing direction during the hybridization reaction according to the second embodiment of the present invention;

FIG. 3A is an exploded view of the biochip according to the third embodiment of the present invention;

FIG. 3B is a cross-section view after the biochip is assembled according to the third embodiment of the present invention;

FIG. 3C is a top view after the biochip is assembled according to the third embodiment of the present invention;

FIG. 3D is a schematic view of the solution flowing direction during the hybridization reaction according to the third embodiment of the present invention;

FIG. 4A is an exploded view of the biochip according to the fourth embodiment of the present invention;

FIG. 4B is a cross-section view after the biochip is assembled according to the fourth embodiment of the present invention;

FIG. 4C is a top view after the biochip is assembled according to the fourth embodiment of the present invention; and

FIG. 4D is a schematic view of the solution flowing direction during the hybridization reaction according to the fourth embodiment of the present invention.

### DETAILED DESCRIPTION OF THE PREFERRED EMBODIMENT

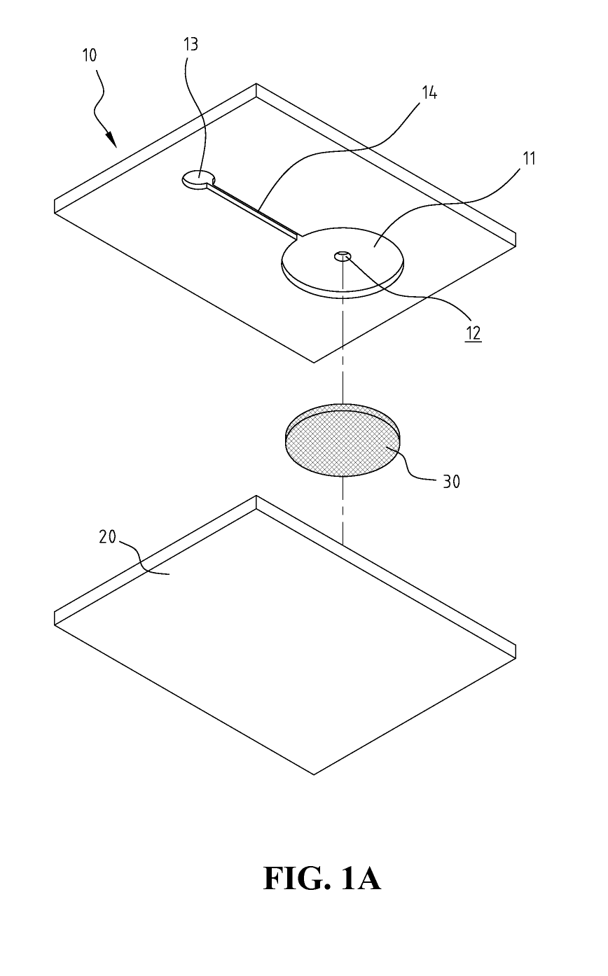

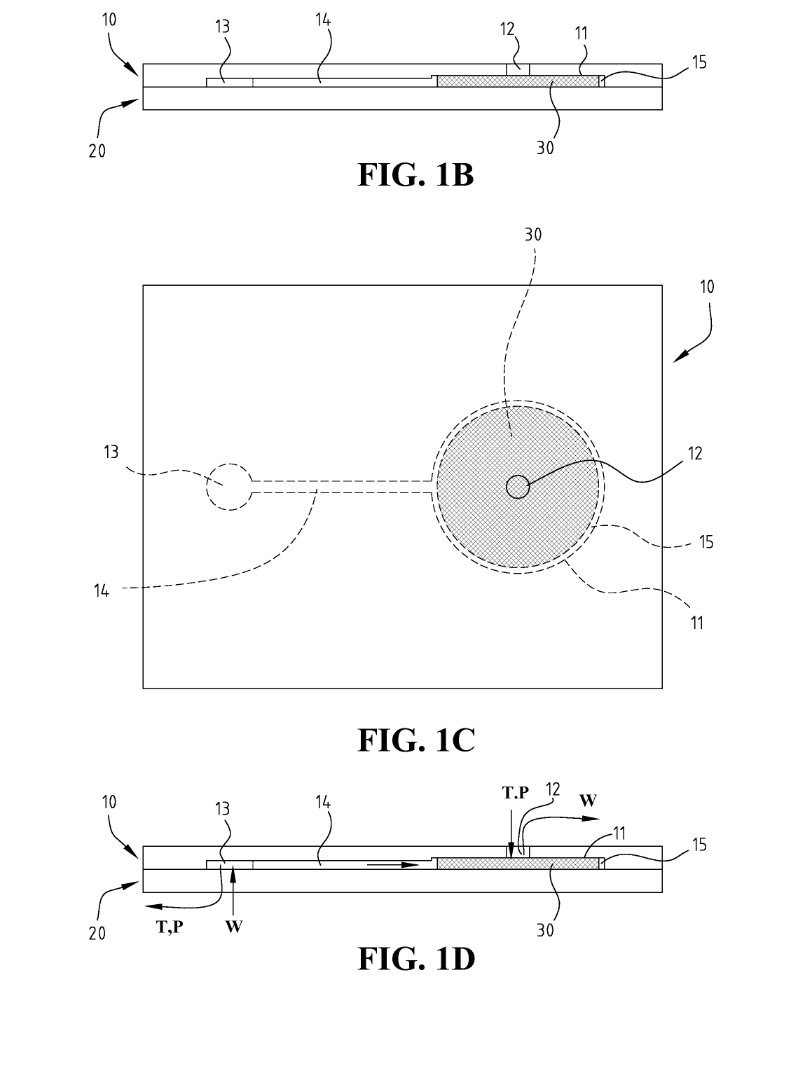

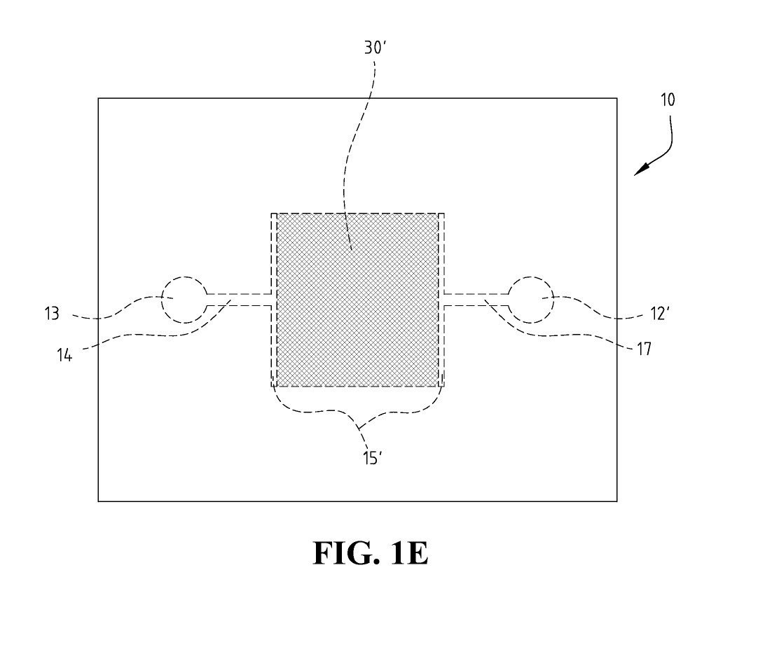

FIG. 1A is an exploded view of the biochip of the first embodiment of the present invention. With reference to FIG. 1A, the biochip of this embodiment comprises an upper substrate 10, a lower substrate 20, and a membrane 30, wherein the upper substrate 10 and the lower substrate 20 are stacked together, and the membrane 30 is provided in the hybridization chamber 11 on the upper substrate 10.

With reference to FIG. 1A, FIG. 1C, and FIG. 1E, the upper substrate 10 has a hybridization chamber 11, which is in the form of a disk-shaped cavity. However, the shape, the size and the thickness of the hybridization chamber 11 have no restriction, and the hybridization chamber 11 can be a tetrahedral cavity (as shown in FIG. 1E). The center of hybridization chamber 11 is provided with a first circulation hole 12. There is no specific limitation on the position of the first circulation hole 12, and the position of the first circulation hole 12 can be changed with the position of another circulation hole so that the reaction solution can flows over the whole inside of the membrane 30. As FIG. 1A shows, the first circulation hole 12 is located at the upper substrate and on the top of the hybridization chamber 11, whereas in FIG. 1E the first circulation hole 12′ may either be located on the side of the hybridization chamber 11. Besides, there is also no specific limitation on the number of the first circulation holes 12. The first circulation hole 12 can be further communicated with a microchannel (such as the microchannel 17 communicated with one side of the hybridization chamber 11 shown in FIG. 1E) or another circulation hole (not shown in the drawings) for facilitating solution injection.

The first microchannel 14 is communicated with one side of the hybridization chamber 11, and the first microchannel 14 is further communicated with the second circulation holes 13. There is no specific limitation on the number and the positions of the first microchannels 14, and the number and the positions thereof can be changed with the flow path of the reaction solution. In addition, an interstice 15 with predetermined width is left between the membrane 30 and the sidewall of the hybridization chamber 11. The interstice 15 has a width of 0.05 to 0.2 mm, and preferably 0.1 mm. In another example, two interstices 15′ with predetermined width, as shown in FIG. 1E, are respectively left between the membrane 30′ and the sidewall of the hybridization chamber 11, and not communicated.

With reference to FIG. 1A and FIG. 1B, the hybridization chamber 11, the first microchannel 14, and the second circulation hole 13 are formed between the upper substrate 10 and the lower substrate 20. The hybridization chamber 11, the first microchannel 14, and the second circulation holes 13 are not limited to be located on the upper substrate 10, but they may be located on the lower substrate 20, or on both the upper substrate 10 and the lower substrate 20 (divided into male and female halves). The upper substrate 10 and the lower substrate 20 may be separately manufactured, or integrally manufactured, and the hybridization chamber 11, the first microchannel 14, and the second circulation hole 13 will be formed inside the substrates while manufactured.

The biochip of this embodiment includes, but not limited to, a microfluidic chip, a nanofluidic chip, or any other structure which is suitable to the present invention. The quartz, glass, or the like can be used as the substrate of the microfluidic chip, and the capillary microchannels are formed by wet etching, and a layer of quartz, or glass covers the tops of the capillary microchannels, and then the chip with the closed microchannels or cavities is produced. Alternatively, the substrate of the biochip is made of the hard polymer, for example, polymethyl methacrylate (PMMA), polycarbonate (PC). First, the mother mold is made by wet-etching the quartz, and then the microchannels are formed on the PMMA or PC material by the embossing method, and then the tops of the microchannels is covered with the same material as the substrate. The substrate of the biochip of the present invention can also be made of soft polymer, for example, polydimethyl siloxane (PDMS). Because of good flowing ability, the thermal compression is not needed to be used so that the mother mold will not be easily damaged, and the biochips can be manufactured in large scale. This makes PDMS a preferable material to be used.

The membrane 30 can be a nylon membrane, a nitrocellulose membrane, or any other suitable membrane fitted in shape for the hybridization chamber 11. The nylon membrane is positive charged or neutral. The nylon membrane and nitrocellulose membrane have a pore diameter of 0.1 μm to 0.5 μm. The proper porous diameter is selected based on the molecular weight of the target nucleic acid, and the larger the nucleic acid, the larger the porous diameter is used. The pore diameter is preferably 0.2 μm to 0.45 μm. Moreover, the membrane can be in a dry state so that the injected target nucleic acid can be adsorbed on the membrane rapidly.

FIG. 1D is the schematic view showing the flow direction of the hybridization solution. With reference to FIG. 1D, the target nucleic acid solution T is injected into the hybridization chamber 11 via the first circulation hole 12, and enters the inside of the membrane 30 from the center thereof. After entering the inside of the membrane 30, the target nucleic acid solution T diffuses across the membrane 30 to the interstice 15 which surrounds the periphery of the membrane 30. Then, the target nucleic acid solution T flows through the first microchannel 14, and is discharged to the outside via the second circulation hole 13. If the membrane 30 is in a dry state, the target nucleic acid solution T can rapidly enter the inside of the membrane 30 due to the capillary attraction of the micropores of the membrane 30. Moreover, the target nucleic acid solution T which enters the membrane 30 can be permanently attached to the surface and the inside of the membrane 30 by heating or UV irradiation.

Afterwards, the nucleic acid probe solution P is injected into the hybridization chamber 11 via the first circulation hole 12 for hybridization reaction. The nucleic acid probe is labeled with chromogenic molecules in order to detect the results of hybridization reaction. The nucleic acid probes can be detectably labeled, for example, with a radioisotope, a fluorescent compound, or an enzyme. The added nucleic acid probe solution P will take the same flow path as the target nucleic acid solution T, and distribute over the whole membrane 30. If the membrane 30 is in a dry state before the nucleic acid probe solution P is added, the nucleic acid probe solution P can also rapidly enter the inside of the membrane 30. After the nucleic acid probe solution P is added, the biochip is placed at a proper temperature (such as 40 to 48 ° C.) for several minutes to allow the nucleic acid probe to anneal with the target nucleic acid, and thus the process of base pairing is completed.

After the process of base pairing, the unhybridized nucleic acid probes are washed away. The washing solution W is injected via the second circulation hole 13 during the washing process. The washing solution W is flushed into the hybridization chamber 11 through the first microchannel 14. When the washing solution W is flushed into the hybridization chamber 11, the interstice 15 is firstly filled with the washing solution, and then the washing solution W diffuses toward the center of the membrane 30 from the edge of the membrane 30, and finally is discharged to the outside via the first circulation hole 12. Because the washing solution W is flushed from the outside of the membrane 30 to the inside of the membrane 30, the nucleic acid probe, which is a relatively small molecule, can be easily and rapidly flushed out of the pores of the membrane 30. Therefore, the background noise level is reduced, and the time for flushing is shortened. Then, the nucleic acid probes labeled with chromogenic molecules are detected, and the results of hybridization reaction are obtained.

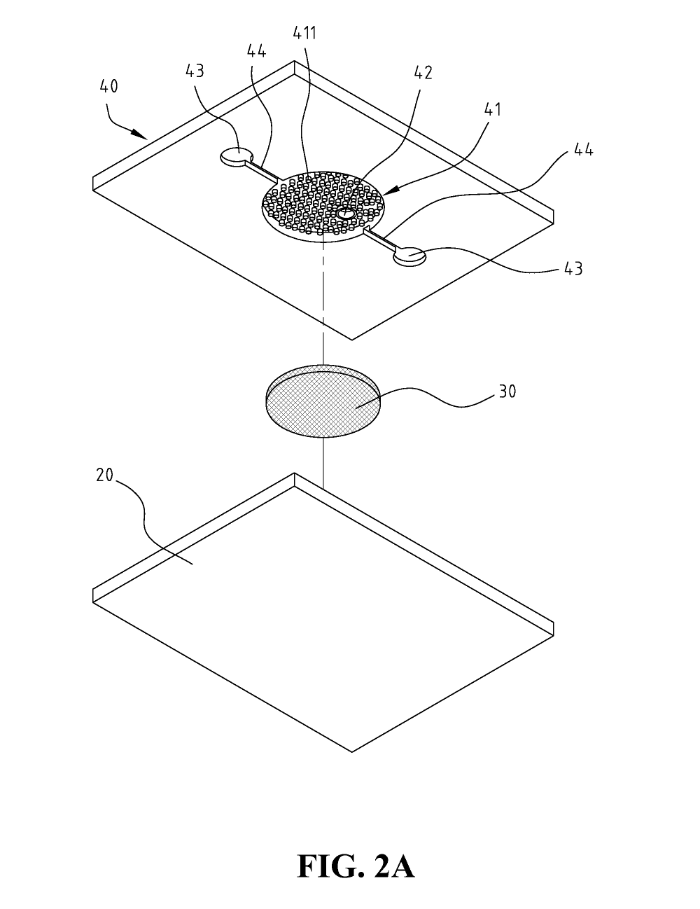

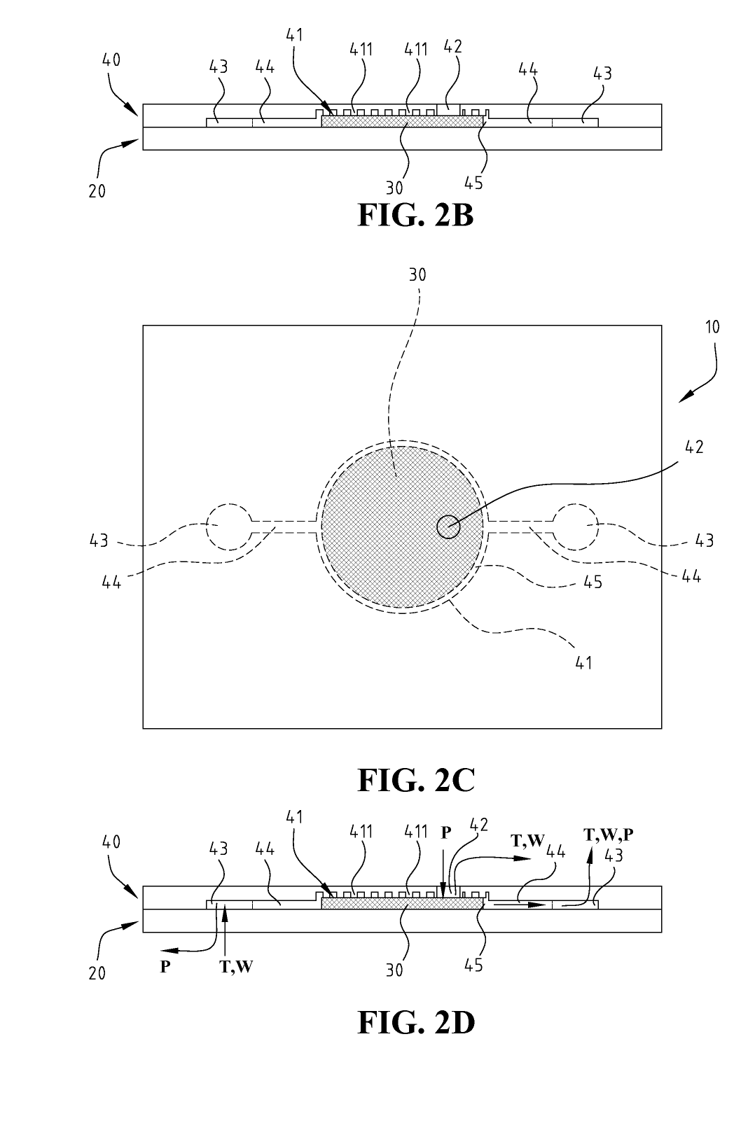

FIG. 2A is an exploded view of the biochip of the second embodiment of the present invention. With reference to FIG. 2A, the biochip of this embodiment comprises an upper substrate 40, a lower substrate 20, and a membrane 30, wherein the upper substrate 40 and the lower substrate 20 are stacked together one on top of the other, and the membrane 30 is provided in the hybridization chamber 41 located on the upper substrate 40.

With reference to FIG. 2A to FIG. 2C, the upper substrate 40 has a hybridization chamber 11, which is in the form of a disk-shaped cavity. However, the shape, the size and the thickness of the hybridization chamber 41 have no restriction, and the hybridization chamber 41 can be a tetrahedral cavity. The center of hybridization chamber 41 is provided with a first circulation hole 42. There is no specific limitation on the numbers and the positions of the first circulation hole 42, and the positions of the first circulation hole 42 can be changed with the position of another circulation hole so that the reaction solution can flows over the whole inside of the membrane 30. In addition, the first circulation hole 42 can be further communicated with a microchannel or another circulation hole (not shown in the drawing) for facilitating the solution injection.

A plurality of little pillars 411 protrude from the interface between the bottom of the upper substrate 40 and the hybridization chamber 41. The ends of these little pillars 411 are in contact with the surface of the membrane 30 located in the hybridization chamber after the biochip is assembled.

A pair of the first microchannel 44 are respectively communicated with the hybridization chamber 41, and the pair of the first microchannel 44 are further respectively communicated with a pair of the second circulation holes 43. There is no specific limitation on the number and the positions of the first microchannel 44, and the number and the positions thereof can be changed with the flow path of the reaction solution. The second circulation holes 43 and the first microchannels 44 are communicated with the interspace among the little pillars 411. In addition, an interstice 45 with predetermined width is left between the membrane 30 and the sidewall of the hybridization chamber 41. The interstice 45 has a width of 0.05 to 0.2 mm, and preferably 0.1 mm.

With reference to FIG. 2A and FIG. 2B, the hybridization chamber 41, the first microchannels 44, and the second circulation hole 43 are formed between the upper substrate 40 and the lower substrate 20. The hybridization chamber 41, the first microchannels 44, and the second circulation holes 43 are not limited to be located on the upper substrate 40, but they may be located on the lower substrate 20, or on the upper substrate 40 and the lower substrate 20 (divided into male and female halves). Once the upper substrate 40 and the lower substrate 20 are stacked together one on top of the other, the desired structures of the hybridization chamber, the microchannels, and the circulation holes will be formed.

The biochips can be fabricated by the conventional method. There is no specific limitation on the material, the shape, and the pore size of the membrane 30.

FIG. 2D is the schematic view showing the flow direction of the hybridization solution according to the second embodiment. With reference to FIG. 2D, before the hybridization reaction, the target nucleic acid solution T is injected into the hybridization chamber 41 from the second circulation hole 43 on the left side of the hybridization chamber through the first microchannels 44. The target nucleic acid solution T firstly fills up the interstice 45, and then enters the inside of the membrane 30 from the lateral side of the membrane 30. While entering the membrane 30 from the lateral side thereof, the target nucleic acid solution T fills up the interspace among the little pillars 411 and then diffuses from the top side the membrane 30 to the bottom side thereof. Finally, the target nucleic acid solution T flows through first microchannels 44 on the right side of the hybridization chamber, and is discharged to the outside via the second circulation hole 43. and the first circulation hole 42. If the membrane 30 is in a dry state, the target nucleic acid solution T can rapidly enter the inside of the membrane 30 due to the capillary attraction of the fine pores of the the membrane 30. Moreover, the target nucleic acid solution T which enters the membrane 30 can be permanently attached to the surface and the inside of the membrane 30 by heating or UV irradiation.

Afterwards, the nucleic acid probe P is injected into the hybridization chamber 41 via the first circulation hole 42 for hybridization reaction. After the nucleic acid probe solution P is added, the added nucleic acid probe solution P will take the same flow path as the target nucleic acid solution T as exemplified in the first embodiment, and distribute over the whole membrane 30. If the membrane 30 is in a dry state before the nucleic acid probe solution P is added , the nucleic acid probe solution P will rapidly enter the inside of the membrane 30. After the nucleic acid probe solution P is added, the biochip is placed at a proper temperature (such as 40 to 48° C.) for several minutes to allow the nucleic acid probe to anneal with the target nucleic acid, and thus the process of base pairing is completed.

After the process of base pairing, the unhybridized nucleic acid probes are washed away. The washing solution W is flushed into the hybridization chamber 41 from the the second circulation hole 43 on the left side through the first microchannel 44. When the washing solution W is flushed into the hybridization chamber 41, the washing solution W diffuses toward the center and the bottom of the membrane 30 from the lateral side and the top side of the membrane 30, respectively, and finally is discharged to the outside through the first circulation hole 42 and the second circulation hole 43 on the right side. Because the washing solution W is flushed from the lateral side and the top side of the membrane 30 to the inside of the membrane 30, the nucleic acid probe, which is a relatively small molecule, can be easily and rapidly flushed out of the pores of the membrane 30. Therefore, the background noise level is reduced, and the time for flushing is shortened.

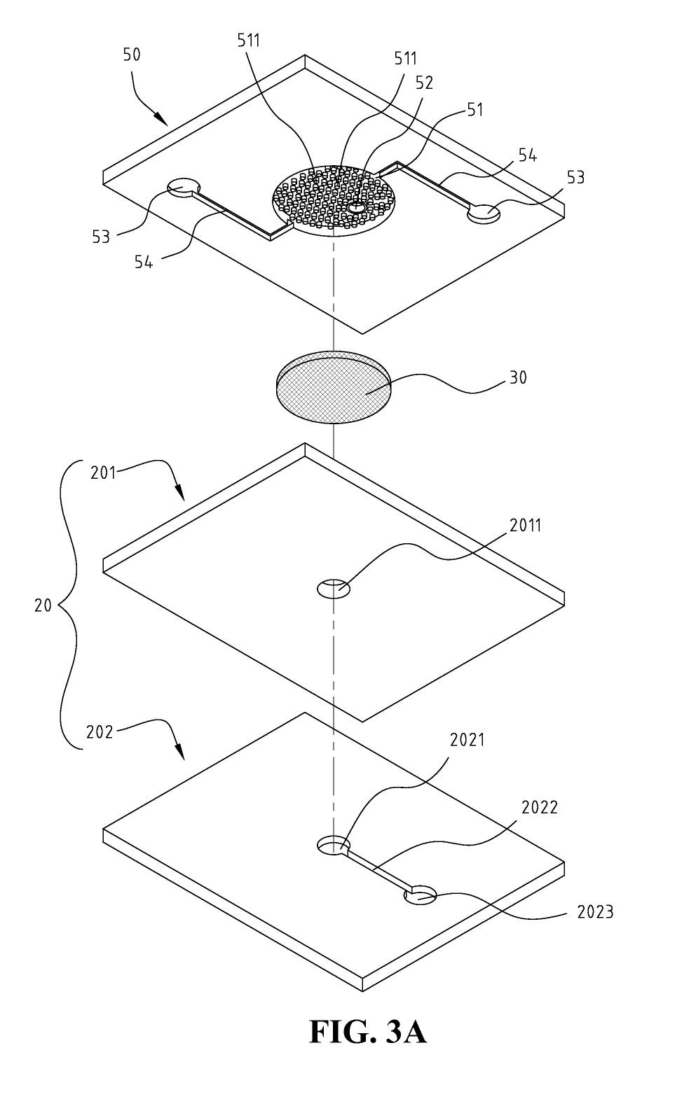

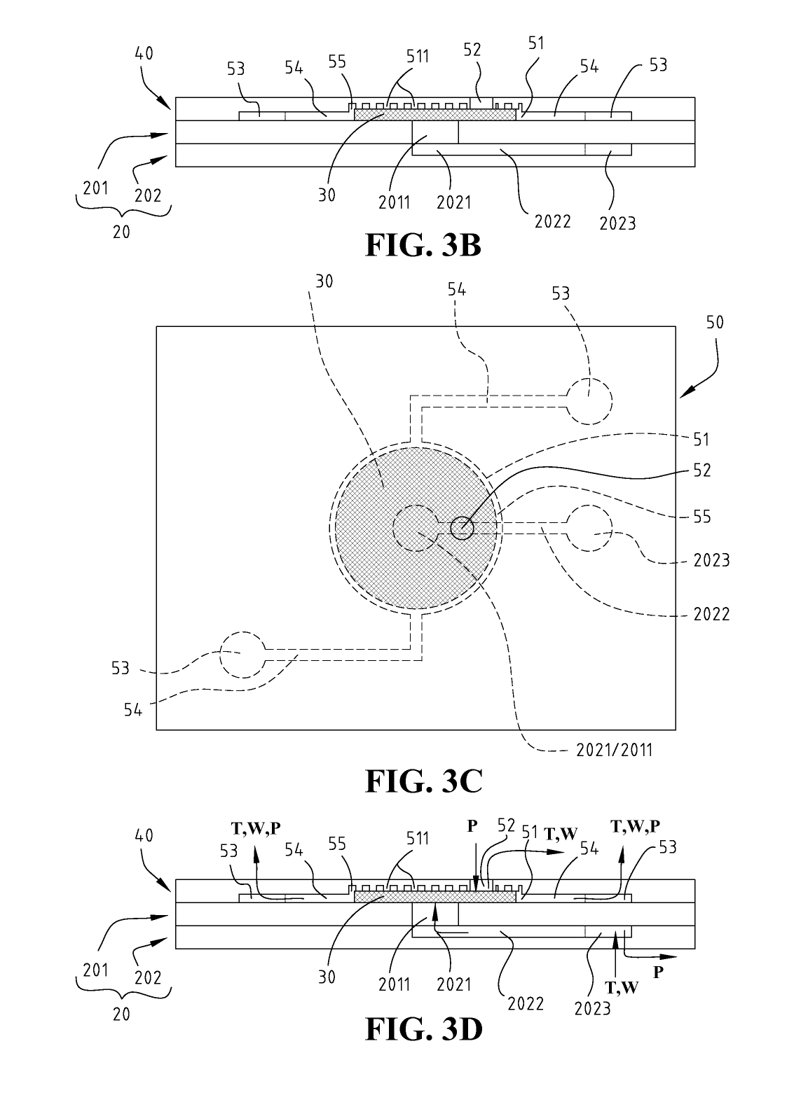

FIG. 3A is an exploded view of the biochip of the third embodiment of the present invention. With reference to FIG. 3A, the biochip of this embodiment comprises an upper substrate 50, a lower substrate 20 composed of a top substrate 201 and a bottom substrate 202, and a membrane 30, wherein the upper substrate 50, the top substrate 201, and the bottom substrate 202 are stacked together one on top of the other, and the membrane 30 is provided in the hybridization chamber 41 located on the upper substrate 50.

With reference to FIG. 3A to FIG. 3C, the upper substrate 40 has a hybridization chamber 51, which is in the form of a disk-shaped cavity. However, the shape, the size and the thickness of the hybridization chamber 41 have no restriction, and the hybridization chamber 41 can be a tetrahedral cavity. The hybridization chamber 41 is provided with a first circulation hole 52. There is no specific limitation on the numbers and the positions of the first circulation hole 52, and the positions of the first circulation hole 52 can be changed with the position of another circulation hole so that the reaction solution can flows over the whole inside of the membrane 30. In addition, the first circulation hole 52 can be further communicated with a microchannel or another circulation hole (not shown in the drawing) for facilitating solution injection.

A plurality of little pillars 511 protrude from the interface between the bottom of the upper substrate 50 and the hybridization chamber 51. The ends of these little pillars 511 are in contact with the surface of the membrane 30 located in the hybridization chamber after the biochip is assembled.

A pair of the first microchannel 54 are respectively communicated with the hybridization chamber 51, and the pair of first microchannels 54 are further respectively communicated with a pair of the second circulation holes 53. There is no specific limitation on the number and the positions of the first microchannels 54, and the number and the positions thereof can be changed with the flow path of the reaction solution. The second circulation holes 53 and the first microchannels 54 are communicated with the interspace among the little pillars 411. In addition, an interstice 55 with predetermined width is left between the membrane 30 and the sidewall of the hybridization chamber 51. The interstice 55 has a width of 0.05 to 0.2 mm, and preferably 0.1 mm.

The lower substrate 20 is composed of a top substrate 201 and a bottom substrate 202, which are stacked together one on top of the other. The top substrate 201 has the third circulation hole 2011, and the bottom substrate 202 has the third circulation hole 2021 corresponding to the third circulation hole 2011. The third circulation hole 2011 or 2021 can be provided in a single-layer lower substrate 20 or in a lower substrate 20 composed of a top substrate 201 and a bottom substrate 202 as this embodiment. The third circulation hole 2021 is further communicated with the second microchannel 2022, and the second microchannel 2022 is further communicated with the fourth circulation hole 2023.

With reference to FIG. 3A and FIG. 3B, the hybridization chamber 51, the first microchannels 54, and the second circulation hole 53 are formed between the upper substrate 50 and the lower substrate 20. The hybridization chamber 51, the first microchannels 54, and the second circulation holes 53 are not limited to be located on the upper substrate 50, but they may be located on the lower substrate 20, or on both the upper substrate 50 and the lower substrate 20 (divided into male and female halves). Once the upper substrate 50 and the lower substrate 20 are stacked together one on top of the other, the desired structures of the hybridization chamber 51, the first microchannels 54, and the second circulation holes 53 will be formed. Likewise, the third circulation holes 2011, 2021, the second microchannels 2022, and the fourth circulation holes 2023 are not limited to be located on the top substrate 201, but they may be located on the bottom substrate 202, or on both the top substrate 201 and the bottom substrate 202 (divided into male and female halves).

The biochips can be fabricated by the conventional method. There is no specific limitation on the material, the shape, and the pore size of the membrane 30.

FIG. 3D is the schematic view showing the flow direction of the hybridization solution according to the third embodiment. With reference to FIG. 3D, before the hybridization reaction, the target nucleic acid solution T is injected into the hybridization chamber 51 from the fourth circulation hole 2023 through the second microchannel 2022, and then the third circulation holes 2021 and 2011. After entering the hybridization chamber 51, the target nucleic acid solution T diffuses into the inside of the membrane 30 from the bottom center thereof, and continuously diffuses toward the outer edge of the membrane 30. Some of the target nucleic acid solution T flows in the interspace among the little pillars 511, and is collected in the interstice 55 surrounding the periphery of the membrane 30. Finally, the target nucleic acid solution T is discharged to the outside via the first microchannel 54 and the second circulation holes 53 which are located on the two sides of the hybridization chamber 51, and the first circulation hole 52. If the membrane 30 is in a dry state, the target nucleic acid solution T can rapidly enter the inside of the membrane 30 due to the capillary attraction of the fine pores of the the membrane 30. Moreover, the target nucleic acid solution T which enters the membrane 30 can be permanently attached to the surface and the inside of the membrane 30 by heating or UV irradiation.

Afterwards, the nucleic acid probe solution P is injected into the hybridization chamber 51 via the first circulation hole 52 for hybridization reaction. After the nucleic acid probe solution P is added, the added nucleic acid probe solution P will take the same flow path as the target nucleic acid solution T as exemplified in the first embodiment, and distribute over the whole membrane 30. If the membrane 30 is in a dry state before the nucleic acid probe solution P is added, the nucleic acid probe solution P will rapidly enter the inside of the membrane 30. After the nucleic acid probe solution P is added, the biochip is placed at a proper temperature (such as 40 to 48° C.) for several minutes to allow the nucleic acid probe to anneal with the target nucleic acid, and thus the process of base pairing is completed.

After the process of base pairing, the unhybridized nucleic acid probes are washed away. The washing solution W is flushed into the hybridization chamber 51 from the fourth circulation hole 2023 through the second microchannel 2022, and then the third circulation holes 2021 and 2011. When the washing solution W is flushed into the hybridization chamber 51, the washing solution W diffuses into the inside of the membrane 30 from the bottom center thereof, and continuously diffuses toward the outer edge of the membrane 30. Some of the washing solution W flows in the interspace among the little pillars 511, and is collected in the interstice 55 surrounding the periphery of the membrane 30. Finally, the washing solution W is discharged to the outside via the first microchannels 54 and the second circulation holes 53 which are located on the two sides of the hybridization chamber 51, and the first circulation hole 52. Because the washing solution W is flushed from the bottom of the membrane 30 to the inside thereof, the nucleic acid probe, which is a relatively small molecule, can be easily and rapidly flushed out of the pores of the membrane 30 by the outward diffusion of the washing solution W and the guidance of the interspace among the little pillars. Therefore, the background noise level is reduced, and the time for flushing is shortened.

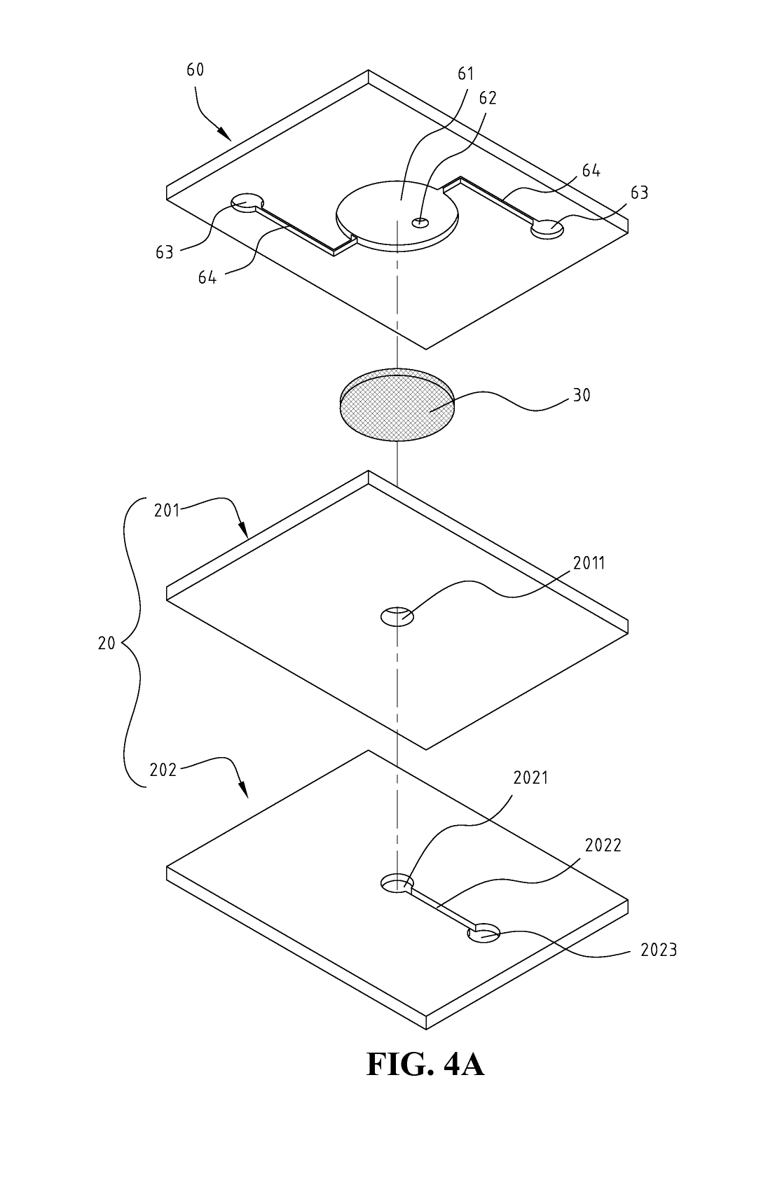

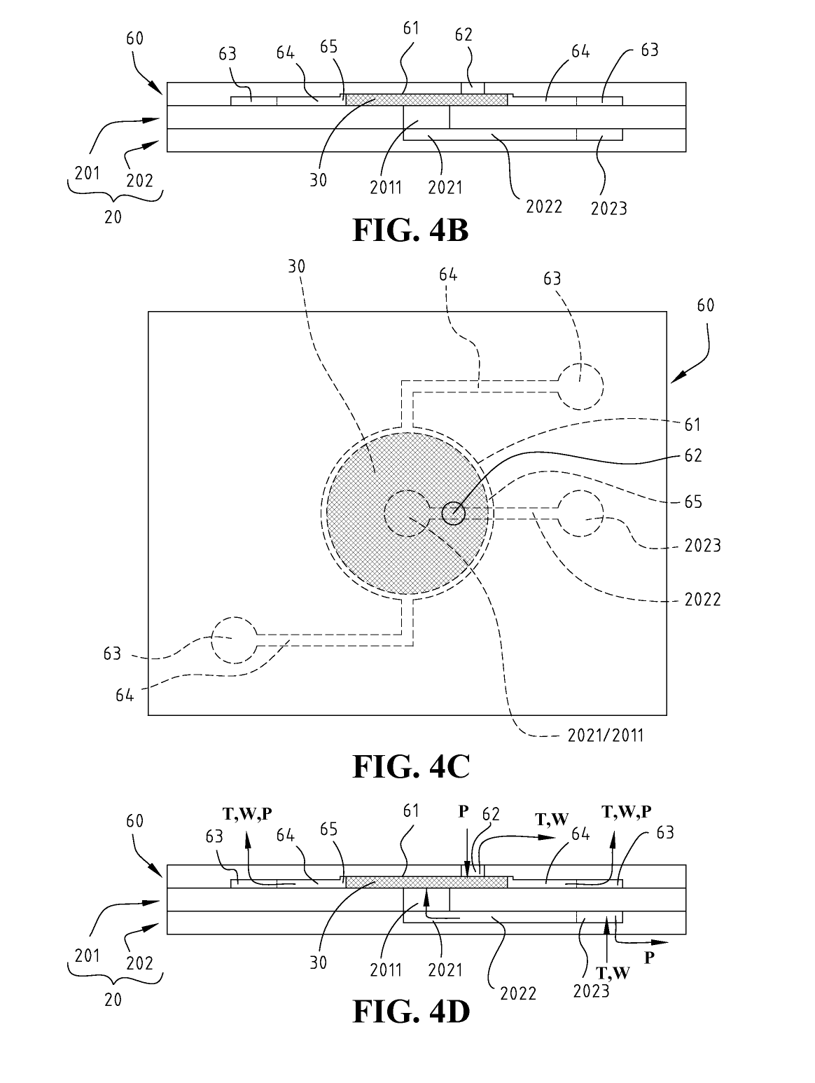

FIG. 4A is an exploded view of the biochip of the fourth embodiment of the present invention. With reference to FIG. 4A, the biochip of this embodiment comprises an upper substrate 60, a lower substrate 20 composed of a top substrate 201 and a bottom substrate 202, and a membrane 30, wherein the upper substrate 60, the top substrate 201, and the bottom substrate 202 are stacked together one on top of the other, and the membrane 30 is provided in the hybridization chamber 61 located on the upper substrate 60.

With reference to FIG. 4A to FIG. 4C, the upper substrate 60 has a hybridization chamber 61, which is in the form of a disk-shaped cavity. However, the shape, the size and the thickness of the hybridization chamber 61 have no restriction, and the hybridization chamber 61 can be a tetrahedral cavity. The hybridization chamber 61 is provided with a first circulation hole 62. There is no specific limitation on the numbers and the positions of the first circulation hole 62, and the positions of the first circulation hole 62 can be changed with the position of another circulation hole so that the reaction solution can flows over the whole inside of the membrane 30. In addition, the first circulation hole 62 can be further communicated with a microchannel or another circulation hole (not shown in the drawing) for facilitating solution injection.

A pair of the first microchannel 64 are respectively communicated with the hybridization chamber 61, and the pair of first microchannels 64 are further respectively communicated with a pair of the second circulation holes 63. There is no specific limitation on the number and the positions of the first microchannels 64, and the number and the positions thereof can be changed with the flow path of the reaction solution. In addition, an interstice 65 with predetermined width is left between the membrane 30 and the sidewall of the hybridization chamber 61. The interstice 65 has a width of 0.05 to 0.2 mm, and preferably 0.1 mm.

The lower substrate 20 is composed of a top substrate 201 and a bottom substrate 202, which are stacked together one on top of the other. The top substrate 201 has the third circulation hole 2011, and the bottom substrate 202 has the third circulation hole 2021 corresponding to the third circulation hole 2011. The third circulation hole 2011 or 2021 can be provided in a single-layer lower substrate 20 or in a lower substrate 20 composed of a top substrate 201 and a bottom substrate 202 as this embodiment. The third circulation hole 2021 is further communicated with the second microchannel 2022, and the second microchannel 2022 is further communicated with the fourth circulation hole 2023.

With reference to FIG. 4 A and FIG. 4 B, the hybridization chamber 61, the first microchannels 64, and the second circulation hole 63 are formed between the upper substrate 60 and the lower substrate 20. The hybridization chamber 61, the first microchannels 64, and the second circulation holes 63 are not limited to be located on the upper substrate 60, but they may be located on the lower substrate 20, or on both the upper substrate 60 and the lower substrate 20 (divided into male and female halves). Once the upper substrate 60 and the lower substrate 20 are stacked together one on top of the other, the desired structures of the hybridization chamber 61, the first microchannels 64, and the second circulation holes 63 will be formed. Likewise, the third circulation holes 2011, 2021, the second microchannels 2022, and the fourth circulation holes 2023 are not limited to be located on the top substrate 201, but they may be located on the bottom substrate 202, or on both the top substrate 201 and the bottom substrate 202 (divided into male and female halves).

The biochips can be fabricated by the conventional method. There is no specific limitation on the material, the shape, and the pore size of the membrane 30.

FIG. 4D is the schematic view showing the flow direction of the hybridization solution according to the fourth embodiment. With reference to FIG. 4D, before the hybridization reaction, the target nucleic acid solution T is injected into the hybridization chamber 61 from the fourth circulation hole 2023 through the second microchannel 2022, and then the third circulation holes 2021 and 2011. After entering the hybridization chamber 61, the target nucleic acid solution T diffuses into the inside of the membrane 30 from the bottom center thereof, and continuously diffuses toward the outer edge of the membrane 30. Subsequently, the target nucleic acid solution T is collected in the interstice 65 surrounding the periphery of the membrane 30. Finally, the target nucleic acid solution T is discharged to the outside via the first microchannels 64 and the second circulation holes 63 which are located on the two sides of the hybridization chamber 51, and the first circulation hole 62. If the membrane 30 is in a dry state, the target nucleic acid solution T can rapidly enter the inside of the membrane 30 due to the capillary attraction of the fine pores of the the membrane 30. Moreover, the target nucleic acid solution T which enters the membrane 30 can be permanently attached to the surface and the inside of the membrane 30 by heating or UV irradiation.

Afterwards, the nucleic acid probe solution P is injected into the hybridization chamber 61 via the first circulation hole 62 for hybridization reaction. After the nucleic acid probe solution P is added, the added nucleic acid probe solution P will take the same flow path as the target nucleic acid solution T as exemplified in the first embodiment, and distribute over the whole membrane 30. If the membrane 30 is in a dry state before the nucleic acid probe solution P is added, the nucleic acid probe solution P will rapidly enter the inside of the membrane 30. After the nucleic acid probe solution P is added, the biochip is placed at a proper temperature (such as 40 to 48° C.) for several minutes to allow the nucleic acid probe to anneal with the target nucleic acid, and thus the process of base pairing is completed.

After the process of base pairing, the unhybridized nucleic acid probes are washed away. The washing solution W is flushed into the hybridization chamber 61 from the fourth circulation hole 2023 through the second microchannel 2022, and then the third circulation holes 2021 and 2011. When the washing solution W is flushed into the hybridization chamber 61, the washing solution W diffuses into the inside of the membrane 30 from the bottom center thereof, and continuously diffuses toward the outer edge of the membrane 30. Subsequently, the washing solution W is collected in the interstice 65 surrounding the periphery of the membrane 30. Finally, the washing solution W is discharged to the outside via the first microchannels 64 and the second circulation holes 63 which are located on the two sides of the hybridization chamber 61, and the first circulation hole 52. Because the washing solution W is flushed from the bottom of the membrane 30 to the inside thereof, the nucleic acid probe, which is a relatively small molecule, can be easily and rapidly flushed out of the pores of the membrane 30. Therefore, the background noise level is reduced, and the time for flushing is shortened.

According to polymerase chain reaction (PCR), the annealing of the primer and the target nucleic acid only took one minute to be completed. By using the biochip of the present invention, the nucleic acid probe can effectively diffuse on part of the surface and in the inside of the membrane and forms a base pair with the target nucleic acid in a very short time after the nucleic acid probe enters the hybridization chamber and contacts with the membrane. The hybridization reaction of the present invention only takes several minutes instead of over 10 hours for the prior art. On the other hand, the nucleic acid probe cannot easily stick to the membrane because the nucleic acid probe can form a base pair with the target nucleic acid in a very short time. Therefore, when the washing solution W is flushed into the inside of the membrane 30, the small nucleic acid probe molecules unspecifically bound to the membrane can be easily and rapidly flushed out of the membrane. As a result, the background noise level is reduced. Moreover, the structures of the flow-in and the flow-out circulation holes for the target nucleic acid solution T, the nucleic acid probe solution P, and the washing solution W described in the above embodiments are just exemplified, therefore, those are not limited to the described ones.

It will be apparent to those skilled in the art that various modifications and variations can be made in the present invention without departing from the spirit or scope of the present invention. Thus, it is intended that the present invention cover the modifications and the variations of this invention provided they come within the scope of the appended claims and their equivalents.

What is claimed is:

1. A biochip, comprising:
a hybridization chamber which is in the form of a cavity;
a porous matrix pressed in the hybridization chamber; and
at least one first circulation hole and at least one second circulation hole, which are communicated with the hybridization chamber so that a reaction solution flows in the at least one first circulation hole and flows out the at least one second circulation hole through a plurality of pores of the porous matrix.

2. The biochip of claim 1, wherein the biochip comprises an upper substrate and a lower substrate, which are stacked together one on top of the other to form the hybridization chamber therein, and the porous matrix is provided in the hybridization chamber, and the at least one first circulation hole located at the upper substrate and the at least one second circulation hole, which are communicated with the hybridization chamber, are provided on a top and a side of the hybridization chamber, respectively.

3. The biochip of claim 2, wherein an interstice with predetermined width is left between the porous matrix and a sidewall of the hybridization chamber.

4. The biochip of claim 2, wherein the second circulation hole is communicated with the hybridization chamber through a first microchannel.

5. The biochip of claim 2, wherein the porous matrix has a pore diameter of 0.1 μm to 50 μm.

6. The biochip of claim 2, wherein the porous matrix is in a dry state.

7. The biochip of claim 2, wherein the porous matrix is a nylon or nitrocellulose membrane.

8. The biochip of claim 2, wherein the lower substrate further has a third circulation hole which is communicated with the hybridization chamber.

9. The biochip of claim 8, wherein the lower substrate is composed of a top substrate and a bottom substrate, which are stacked together one on top of the other, and the third circulation hole is provided between the top substrate and the bottom substrate.

10. The biochip of claim 1, wherein the biochip comprises an upper substrate and a lower substrate, which are stacked together one on top of the other to form the hybridization chamber therein, and the porous matrix is provided in the hybridization chamber, and the at least one first circulation hole and the at least one second circulation hole, which are communicated with the hybridization chamber, are provided on sides of the hybridization chamber respectively.

11. A biochip, comprising:
an upper substrate and a lower substrate, which are stacked together one on top of the other;
a hybridization chamber provided between the upper substrate and the lower substrate;
a porous matrix pressed in the hybridization chamber;
a plurality of little pillars protruding from an interface between a bottom of the upper substrate and the hybridization chamber wherein a plurality of ends of the little pillars are in contact with the surface of the porous matrix pressed in the hybridization chamber; and
at least one first circulation hole located at the upper substrate and at least one second circulation hole, the at least one first circulation hole being provided on a top of the hybridization chamber and the at least one second circulation hole being provided at a side of the hybridization chamber, wherein the at least one second circulation hole is communicated with the interspace among the little pillars.

12. The biochip of claim 11, wherein an interstice with predetermined width is left between the porous matrix and a sidewall of the hybridization chamber.

13. The biochip of claim 11, wherein the hybridization chamber has at least one third circulation hole penetrated through the lower substrate.

14. The biochip of claim 13, wherein the lower substrate is composed of a top substrate and a bottom substrate, which are stacked together one on top of the other, and the third circulation hole is provided between the top substrate and the bottom substrate.

15. The biochip of claim 13, wherein the third circulation hole is further communicated with a second microchannel, and the second microchannel is further communicated with a fourth circulation hole.

16. The biochip of claim 11, wherein the first circulation hole is further communicated with the hybridization chamber through a third microchannel.

17. The biochip of claim 11, wherein the porous matrix has a pore diameter of 0.1 μm to 50 μm.

18. The biochip of claim 11, wherein the porous matrix is in a dry state.

19. The biochip of claim 11, wherein the porous matrix is a nylon or nitrocellulose membrane.
